(19) United States
(12) Patent Application Publication
(10) Pub. No.: US 2019/0099236 A1
(43) Pub. Date: Apr. 4, 2019

(54) MAXILLARY TUBEROSITY HOLDING BITE BLOCK

(71) Applicants: Xin Jin, Beijing (CN); Xiaoyu Lu, Covina, CA (US)

(72) Inventors: Xin Jin, Beijing (CN); Xiaoyu Lu, Covina, CA (US)

(21) Appl. No.: 16/137,444

(22) Filed: Sep. 20, 2018

Related U.S. Application Data
(60) Provisional application No. 62/567,436, filed on Oct. 3, 2017.

(51) Int. Cl. A61B 90/16

(52) U.S. Cl. CPC A61B 90/16; A61C 5/007

(57) ABSTRACT

A maxillary tuberosity holding bite block is disclosed. The maxillary tuberosity holding bite block includes a curved tail that embraces a patient's maxillary tuberosity, which allows the bite block to hold the patient's jaws in a stable position, regardless of whether or not the patient has edentulous jaws. Moreover, the maxillary tuberosity holding bite block may serve as a carrier for sensors and endoscopes so it may serve as an intra-oral sensor-based positioning and navigation device that sense and track the movement and position of sensor-equipped surgical instruments.

### RELATED APPLICATION

This application claims the benefit of priority under 35 U.S.C. § 119(e) to Provisional Application No. 62/567,436, filed on Oct. 3, 2017, which is incorporated by reference in its entirety.

### STATEMENT REGARDING FEDERALLY SPONSORED RESEARCH OR DEVELOPMENT

Not applicable

### INCORPORATION BY REFERENCE OF MATERIAL SUBMITTED ON A COMPACT DISC

Not applicable

### THE NAMES OF THE PARTIES TO A JOINT RESEARCH AGREEMENT

Not applicable

### BACKGROUND OF THE INVENTION

Bite blocks are commonly used in oral surgical procedure for preventing patients from closing their jaws during the oral surgical procedure. Traditionally, a bite block is shaped like a wedge placed between a patient's upper jaw and lower jaw in order to maintain a space between them. Traditional bite blocks may shift during the oral surgical procedure, which alters the patient's jaw spatial positions. Although such shifting may be inconsequential for certain oral surgical procedure, maintaining a stable position throughout the entire oral surgical procedure may be crucial in more complex or delicate oral surgical procedure, for example, in oral surgeries using mixed reality technology and intra-oral navigation where accurate positioning is required. The present invention relates to a novel bite block that ensures the patient's jaws remain in a stable spatial position during an oral surgical procedure.

### BRIEF SUMMARY OF THE INVENTION

The present invention discloses a bite block having a curved tail that embraces a patient's maxillary tuberosity, which allows the bite block to hold the patient's jaws in a stable spatial position. Traditional bite blocks incorporate grooves in the bite block. When patients “bite down” onto the bite blocks, the patients' teeth bite onto the groove so that the bite blocks may remain in place. However, this is not an effective way of holding the bite blocks in place for patients who have no teeth, i.e., edentulous jaws. Using a curved-tail bite block that embraces a patient's maxillary tuberosity will eliminate the concern of a patient having edentulous jaws. Moreover, the bite block may serve as a carrier for sensors (optical and/or magnetic) and endoscopes so that it may also serve as an intra-oral sensor-based positioning and navigation device that senses and tracks the movement and position of sensor-equipped surgical instruments.

It is an object of this invention to provide a bite block that holds a patient's jaws in a stable spatial position.

It is a further object of this invention to provide a bite block that may serve as a carrier for sensors, endoscopes, and/or navigation detection systems.

These and other objects are preferably accomplished by providing a maxillary tuberosity bite block comprising a curved tail that embraces a patient's maxillary tuberosity, which allows the bite block to hold the patient's jaws in a stable spatial position. Optionally, the maxillary tuberosity bite block may further include sensors (e.g., optical, magnetic, or electromagnetic) and miniature endoscopes in order to facilitate the use of mixed reality technology.

### BRIEF DESCRIPTION OF THE DRAWINGS

The above-mentioned features and objects of the present disclosure will become more apparent with reference to the following description taken in conjunction with the accompanying drawings wherein like reference numerals denote like elements and in which:

FIGS. 1A to 1C are perspective view of a traditional bite block.

FIG. 1D is a perspective view of a traditional bite block being placed between a patient's upper and lower jaws.

FIG. 2 is a perspective view of an embodiment of the maxillary tuberosity holding bite block of the present invention.

FIGS. 3A and 3B are graphic representation of an embodiment of the maxillary tuberosity holding bite block of the present invention being placed between jaws.

FIG. 4 is an illustration of an embodiment of the maxillary tuberosity holding bite block of the present invention wherein the bite block includes sensors and miniature endoscopes.

### DETAILED DESCRIPTION OF THE INVENTION

For illustrative purpose, the principles of the present invention are described by referring to an exemplary embodiment thereof. Before any embodiment of the invention is explained in detail, it should be understood that the invention is not limited in its application to the details of construction and the arrangement of components set forth in the following description or illustrated in the following drawings. The invention is capable of other embodiments and of being practiced or of being carried out in various ways. Also, it should be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items.

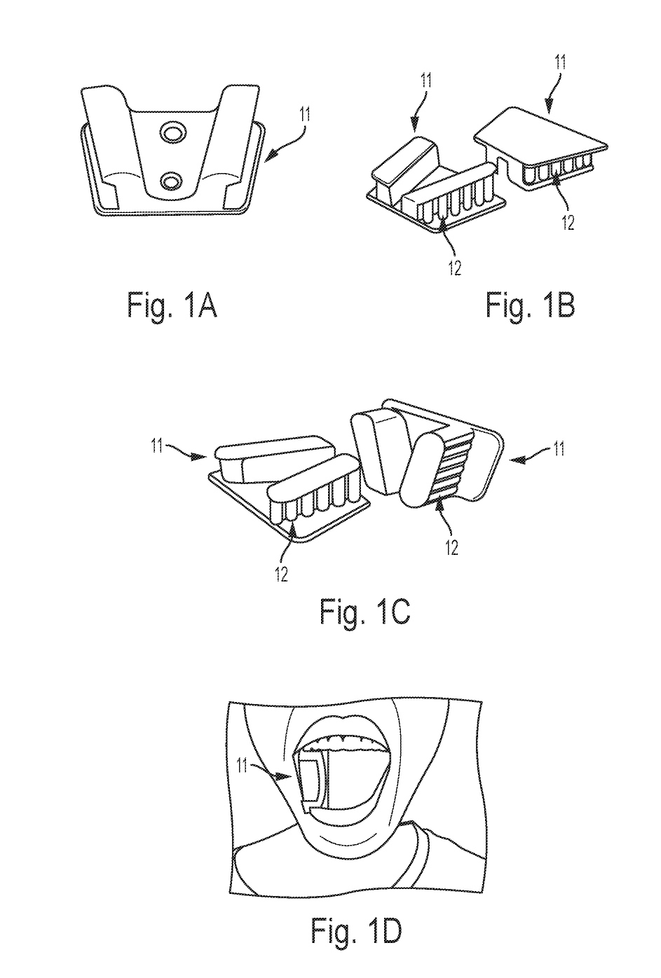

FIGS. 1A, 1B, and 1C illustrate a traditional bite block 11 incorporating grooves 12. When it is placed between the jaws of a patient, as shown in FIG. 1D, the patient bite down onto the bite block 11 and the patent's mouth stays open. Because there is no other means to hold the bite block in place other than the force from the patient's bite, the bite block may move or shift, for example, due to jaw movement by the patient or the patient's edentulous jaws, which may alter the patient's jaw spatial positions.

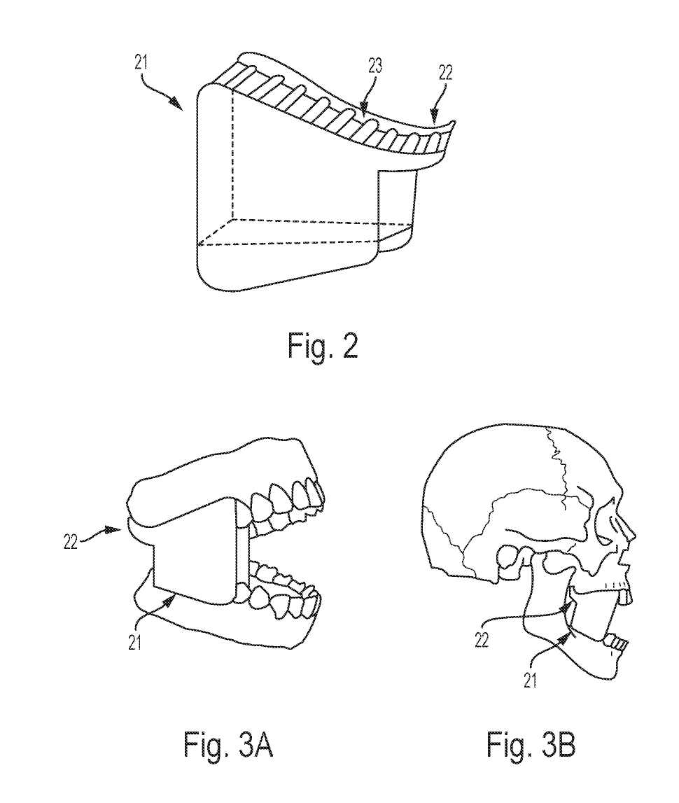

FIG. 2 shows one embodiment of the maxillary tuberosity holding bite block of the present invention. The maxillary tuberosity holding bite block 21 includes a curved tail 22 that embraces the maxillary tuberosity of the patient (as showed in FIGS. 3A and 3B). The curved tail 22 secured the maxillary tuberosity holding bite block in place. With the curved tail 22, the bite block will be secured in place even for patients having edentulous jaws (toothless jaws). In one embodiment, the maxillary tuberosity holding bite block may include an upper surface having one or more grooves 23 that provides more grip when the patient bites down onto the maxillary tuberosity bite block.

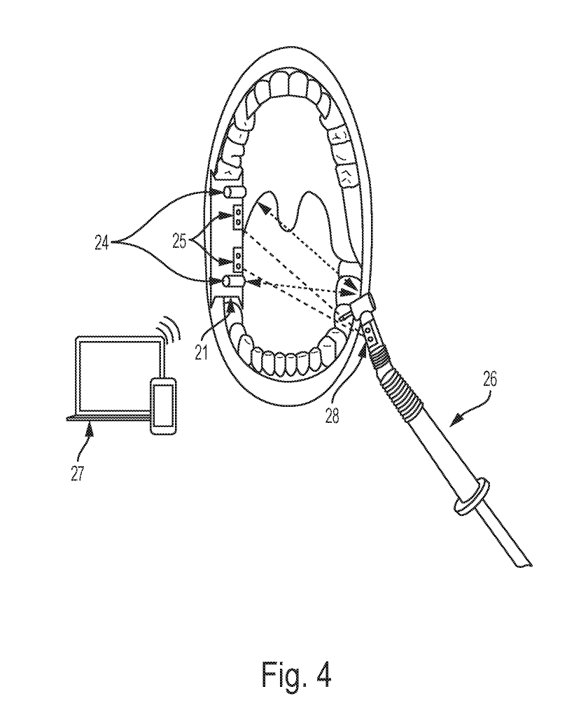

FIG. 4 describes an embodiment of the maxillary tuberosity holding bite block of the present invention wherein the bite block 21 includes sensors 24 and endoscopes 25. In one embodiment, the one or more sensors 24 may be optical, magnetic, and/or electromagnetic. In one embodiment, the sensors 24 sense and track the spatial position a dental instrument 26. The sensors 24 may transmit the spatial position of the dental instrument 26 to a remote system 27 (which may be a portable device such as a laptop computer or mobile phone) for processing and/or viewing.

As illustrated in FIG. 4, the endoscopes 25 of the maxillary tuberosity holding bite block may provide anatomy information of the patient's mouth, including, for example, the tooth that requires dental care, as well as the spatial position of a dental instrument. In one embodiment, the dental instrument 26 may also include endoscopes 28 that provide anatomy information of the patient's mouth. Thus, the sensors 24 and/or endoscopes 25 may be used as an intra-oral sensor-based positioning and navigation device that sense and track the movement and position of a dental instruments. In oral surgeries using mixed reality technology and intra-oral navigation, the maxillary tuberosity holding bite block of the present invention may serve as a carrier for sensors and endoscopes in order to provide accurate information of patients to surgeons.

The previous description is provided to enable any person of ordinary skill in the art to make or use the disclosed methods and apparatus. Various modifications to these examples will be readily apparent to those skilled in the art, and the principles defined herein may be applied to other examples without departing from the spirit or scope of the disclosed method and apparatus. The described embodiments are to be considered in all respects only as illustrative and not restrictive and the scope of the invention is, therefore, indicated by the appended claims rather than by the foregoing description. All changes, which come within the meaning and range of equivalency of the claims, are to be embraced within their scope. Skilled artisans may implement the described functionality in varying ways for each particular application, but such implementation decisions should not be interpreted as causing a departure from the scope of the disclosed apparatus and methods. The steps of the method or algorithm may also be performed in an alternate order from those provided in the examples.

1. A maxillary tuberosity holding bite block comprising:
a wedge-shaped element configured to fit between a patient's upper jaw and lower jaw wherein the wedge shaped element further includes a curved tail configured to embrace the patient's maxillary tuberosity;

2. The maxillary tuberosity holding bite block of claim 1 wherein the wedge-shaped element further comprises an upper surface facing the patient's upper jaw; the upper surface comprises one or more grooves.

3. The maxillary tuberosity holding bite block of claim 1 further comprising one or more sensors.

4. The maxillary tuberosity holding bite block of claim 1 further comprising one or more endoscopes.

5. The maxillary tuberosity holding bite block of claim 3 wherein the one or more sensors are optical, magnetic, or electromagnetic sensors.
